(19) United States
(12) Patent Application Publication
(10) Pub. No.: US 2025/0367081 A1
(43) Pub. Date: Dec. 4, 2025

(54) SINGLE DOSE ORAL HYGIENE UNIT OF TOOTHPASTE COMPOSITION AND METHOD OF MANUFACTURE

(71) Applicant: Poppits LLC, Ft Lauderdale, FL (US)

(72) Inventors: WAYNE R. SOLAN, Ft Lauderdale, FL (US); ALAN DALY, Charlotte, NC (US); SUSAN FOWLER, Braine Le Château (BE)

(73) Assignee: Poppits LLC, Ft Lauderdale, FL (US)

(21) Appl. No.: 18/678,792

(22) Filed: May 30, 2024

(51) Int. Cl. A61K 8/02; A61Q 11/00

(52) U.S. Cl. CPC A61K 8/0216; A61Q 11/00; A61K 2800/622; A61K 2800/92

(57) ABSTRACT

A single-dose oral hygiene unit of a toothpaste composition is blended together using a method of manufacturing disclosed herein. The oral hygiene units define an outer surface that is easily manipulated with a user's fingers. The ingredients are first blended together to create a final, blended toothpaste composition. A predetermined weight of blended toothpaste composition is extruded via nozzles into a plurality of cavities defined in a mold tray. The mold tray containing blended toothpaste composition is refrigerated for a predetermined time at a predetermined temperature until the blended toothpaste composition solidifies and the single-dose oral hygiene units are formed. The units may undergo a coating process whereby the outer surface of each unit is coated with an ingredient that prevents one or more units from sticking to one another or the container in which the units are placed and dispensed from.

### FIELD OF THE INVENTION

The disclosure herein pertains to oral hygiene, and more particularly relates to a single-dose oral hygiene unit of a toothpaste composition and a method of manufacturing the toothpaste composition into single-dose oral hygiene unit.

### DESCRIPTION OF THE PRIOR ART AND OBJECTIVES OF THE INVENTION

See for example, U.S. Pat. No. 8,113,729 granted Feb. 14, 2012, entitled TOOTHPASTE DROPLETS, U.S. Pat. No. 8,827,583 granted Sep. 9, 2014, entitled TOOTHPASTE DROPLETS, U.S. Pat. No. 9,750,669 granted Sep. 5, 2017, entitled TOOTHPASTE DROPLETS, U.S. Pat. No. 10,653,235 granted May 19, 2020, entitled TOOTHPASTE DROPLETS, all of which are assigned to the assignee/applicant of the subject application, each of which are incorporated by reference in their entireties herein.

Excessive ingestion of toothpaste, and other oral hygiene compositions, is not prevented by traditional tubes of toothpaste. It is commonly understood that a pea-sized amount of toothpaste is all that is required to effectively clean and protect teeth. With respect to typical tubes of toothpaste, a pea-sized amount of toothpaste should be squeezed out of the tube onto the bristles of the toothpaste. Furthermore, traditional toothpaste tubes are made of plastic and other non-biodegradable materials and billions of toothpaste tubes are discarded each year and end up in landfills or worse, in the ocean. Innovation is required to eliminate excessive use and ingestion of toothpaste, and to remove the need to use the conventional plastic toothpaste tube.

Thus, in view of the problems and disadvantages associated with prior art devices, the present disclosure was conceived and one of its objectives is to provide a single-dose oral hygiene unit manufactured by a composition that may be delivered and configured in a single-dose oral hygiene unit, wherein multiple single-dose oral hygiene units may be stored together in a container and are convenient for a user to pick up with their fingers and placed into their mouth to begin brushing with a wet toothbrush.

It is also an objective of the present disclosure to provide an oral hygiene unit of toothpaste composition for cleaning teeth, the toothpaste composition may be formed into small shapes, or units, which are convenient to pick up with a user's fingers and place into the mouth prior to brushing with a wet toothbrush.

It is a further objective of the present disclosure to provide an oral hygiene unit of toothpaste composition that comprises a soft, elastic consistency due to the presence of a gumming ingredient in the toothpaste composition, the oral hygiene unit easily dissolves and foams up in the mouth with a wet toothbrush and agitation of the brushing action.

It is another objective of the present disclosure to provide a toothpaste composition that may be formed into a pre-portioned, bite-sized, single-dose oral hygiene unit having a soft, elastic consistency due to the presence of gum in the ingredients, and that is easy to break up in the mouth with a toothbrush.

It is still another objective of the present disclosure to provide a single-dose oral hygiene unit that is surface treated to prevent the oral hygiene units from sticking together and allow them to be easily separated by a user.

It is yet another objective of the present disclosure to provide an improved means for storing and using toothpaste that does not accumulate excess toothpaste buildup around the opening of the toothpaste container.

It is a further objective of the present disclosure to provide a method of blending a toothpaste composition to form a single-dose oral hygiene unit that may be surface treated with a coating ingredient that prevents the oral hygiene units from sticking together and allows them to be easily separated.

It is still a further objective of the present disclosure to provide a method of manufacturing single-dose oral hygiene units by blending a toothpaste composition, extruding predetermined amounts of the blended toothpaste composition into cavities defined on a mold tray, refrigerating the mold tray until the blended toothpaste composition solidifies and the single-dose oral hygiene units are formed, removing the single-dose oral hygiene units from the mold tray, and treating an outer surface of the single-dose oral hygiene units with a coating ingredient that prevents the oral hygiene units from sticking together and allows them to be easily separated.

It is yet a further objective of the present disclosure to provide a method of forming a single-dose oral hygiene unit without an outer membrane.

Various other objectives and advantages of the present disclosure will become apparent to those skilled in the art as a more detailed description is set forth below.

### SUMMARY OF THE INVENTION

The aforesaid and other objectives are realized by providing and blending various ingredients to form a toothpaste composition comprising a gumming ingredient, a surfactant ingredient, a humectant ingredient, an abrasive ingredient, and flavoring ingredient. In certain embodiments, the toothpaste composition may also include an active ingredient for the mitigation, prevention, and/or treatment of cavities, sensitivity, tartar, staining, and/or enamel damage. The toothpaste composition is blended together using a method to be described below and is preferably formed into a single-dose oral hygiene unit. The single-dose oral hygiene unit is an amount of oral care composition to be administered to a patient or user in a single use. The single-dose oral hygiene units are formed without an outer membrane and are quickly workable into a foam when a user inserts the unit into their mouth and brushes the unit with a wet toothbrush. The brushing motion will cause the oral hygiene unit to lather up into a workable foam that can be worked throughout the oral cavity of the user. The outer surface of the single-dose oral hygiene unit is treated with a coating ingredient that prevents the oral hygiene units from sticking together and allows a user to easily and cleanly pick up the unit with their fingers.

The oral hygiene units are formed using a novel method of blending, extruding, refrigerating, and surface treating a toothpaste composition. More specifically, the method includes blending the toothpaste composition by following a specific step-wise set of instructions. After the toothpaste composition is fully blended, a predetermined amount of the blended toothpaste composition is extruded into cavities defined on a mold tray. The mold tray is then refrigerated until the blended toothpaste composition solidifies and the oral hygiene units are formed. The single-dose oral hygiene units are removed from the mold tray and an outer surface of the single-dose oral hygiene units is treated with a coating ingredient that prevents the oral hygiene units from sticking together and allows them to be easily separated and manipulated.

### BRIEF DESCRIPTION OF THE DRAWINGS

FIG. 1 shows a perspective view of a container for dispensing an oral hygiene unit made in accordance with one embodiment of the present invention.

FIG. 2A pictures a perspective view of an oral hygiene unit having a round shape.

FIG. 2B pictures a perspective view of an oral hygiene unit having a hemi-spherical shape.

FIG. 2C pictures a perspective view of an oral hygiene unit having a square shape.

FIG. 2D pictures a perspective view of an oral hygiene unit having a teardrop shape.

FIG. 2E pictures a perspective view of an oral hygiene unit having a lemon wedge shape.

FIG. 2F pictures a perspective view of an oral hygiene unit having a football shape.

FIG. 2G pictures a perspective view of an oral hygiene unit having a heart shape.

FIG. 3 depicts a flow chart detailing the steps for manufacturing an oral hygiene unit.

### DETAILED DESCRIPTION OF THE PREFERRED EMBODIMENT AND OPERATION OF THE INVENTION

Various exemplary embodiments of the present disclosure are described below. Use of the term “exemplary” means illustrative or by way of example only, and any reference herein to “the disclosure” is not intended to restrict or limit the disclosure to exact features or step of any one or more of the exemplary embodiments disclosed in the present specification. References to “exemplary embodiment”, “one embodiment”, “an embodiment”, “various embodiments”, and the like may indicate that the embodiment(s) of the disclosure so described may include a particular feature, structure, or characteristic, but not every embodiment necessarily incudes the particular feature, structure, or characteristic. Further, repeated use of the phrase “in one embodiment”, “in an exemplary embodiment”, or “in an alternative embodiment” do not necessarily refer to the same embodiment, although they may.

It is also noted that terms like “preferably”, “commonly”, and “typically” are not utilized herein to limit the scope of the disclosure or to imply that certain features are critical, essential, or even important to the structure or function of the disclosure. Rather, these terms are merely intended to highlight alternative or additional features that may or may not be utilized in a particular embodiment of the present disclosure.

The disclosure may be embodied in many different forms and should not be construed as limited to the embodiments set forth herein; rather, these embodiments are provided so that this disclosure will be operative, enabling, and complete. Accordingly, the particular arrangements disclosed are meant to be illustrative only and not limited as to the scope of the disclosure, and any and all equivalents thereof. Moreover, many embodiments such as adaptations, variations, modifications, and equivalent arrangements will be implicitly disclosed by the embodiments described herein and fall within the scope of the instant disclosure.

Although specific terms are employed herein, they are used in a generic and descriptive sense only and not for the purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. Where only one item is intended, the terms “one and only one”, “single”, or similar language is used. When used herein to join a list of items, the term “or” denotes at least one of the items but does not exclude a plurality of items of the list.

For exemplary methods or processes of the disclosure, the sequence and/or arrangement of steps described herein are illustrative and not restrictive. Accordingly, it should be understood that, although steps of various processes or methods may be shown and described as being in a sequence or temporal arrangement, the steps of any such processes or methods are not limited to being carried out in any particular sequence or arrangement, absent an indication otherwise. Indeed, the steps in such processes or methods generally may be carried out in various different sequences and arrangements while still falling within the scope of the present disclosure.

Additionally, any references to advantages, benefits, unexpected results, or operability of the present disclosure are not intended as an affirmation that the disclosure has previously been reduced to practice or that any testing has been performed. Likewise, unless stated otherwise, use of verbs in the past tense (present perfect or preterit) is not intended to indicate or imply that the disclosure has previously been reduced to practice or that any testing has been performed.

In the present disclosure, all percentages and ratios are expressed on a weight basis relative to the total composition unless specified otherwise. The percentages, ratios, and concentrations of ingredients detailed herein are calculated based on the pure form of these ingredients, excluding any solvents, fillers, or auxiliary substances that may be present in commercially available formulations of these ingredients, unless indicated otherwise.

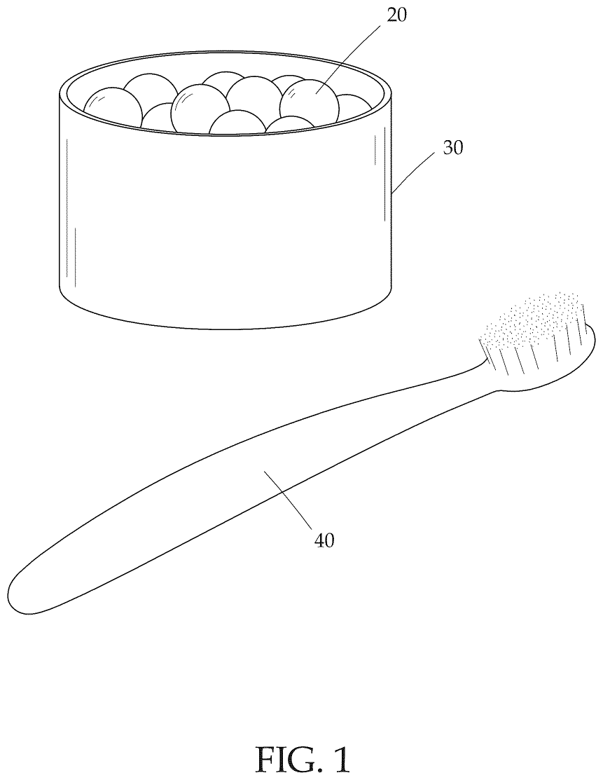

For a better understanding of the disclosure and its operation, turning now to the drawings, FIGS. 1-3 demonstrate a toothpaste composition that may be formed, using a method of manufacture disclosed herein, into a single-dose oral hygiene unit that may be treated with a coating ingredient that facilitates the prevention of oral hygiene units from sticking together and allows the units to be easily separated and manipulated by a user. The toothpaste composition may comprise one or more gumming ingredients, one or more surfactant ingredients, one or more humectant ingredients, one or more abrasive ingredients, and one or more flavoring ingredients. The toothpaste composition may include one or more active ingredients that facilitate the mitigation, prevention, and/or treatment of cavities, tooth sensitivity, tartar buildup, staining, and/or enamel damage. The above mentioned ingredients may be blended together under a set of specific conditions, as will be described in further detail below, allowing the blended toothpaste composition to be formed into homogenous, pre-portioned, bite-sized oral hygiene units that may be packaged together without sticking together and may be easily manipulated by a user. After the ingredients have been properly blended together, the final blended toothpaste composition is extruded into a mold tray containing multiple cavities, which define the shape and size of the resulting oral hygiene units. The cavities defined in the mold tray may take any shape and ideally are formed to contain a predetermined amount of final blended toothpaste composition. Once the cavities are filled with the final blended toothpaste composition, the mold trays are passed through a cooling tunnel to allow the final blended toothpaste composition in each cavity to solidify into a jelly-like toothpaste structure. Once the final blended toothpaste composition has solidified into the single-dose oral hygiene units, the units are removed from the mold tray and undergo a coating step where each unit is treated with a coating ingredient to reduce the likelihood that the oral hygiene units stick together when they are packaged together to allow the single-dose oral hygiene units to exit the container one unit at a time. The specific manufacturing process will be explained in further detail below.

The first step in creating the single-dose oral hygiene units is to properly blend specific ingredients together. The final blended toothpaste composition may include one or more gumming ingredients, capable of binding the ingredients of the toothpaste composition together. In the present disclosure, the one or more gumming ingredients are capable of preventing the separation of the powder and liquid ingredients of the toothpaste composition. The gumming ingredients shall also be capable of creating a smooth texture of the final toothpaste composition and resulting single-dose oral hygiene unit. The gumming ingredients preferably have good water solubility to allow the unit to emulsify and foam up in the presence of water. In the preferred embodiment, the one or more gumming ingredients are selected from the group consisting of carrageenan, xanthan gum, guar gum, arabic gum, carob bean gum, cellulose gum, and combinations thereof. In the most preferred formulation, the gumming ingredient includes carrageenan. In certain embodiments, the amount of gumming ingredient exists in a range of about 0.5% to about 5.0% by weight of the total toothpaste composition. The more preferred range of gumming ingredient present is about 1.25% to about 2.5% by weight of the total toothpaste composition. The most preferred amount of gumming ingredient present in the final toothpaste composition is about 1.5% by weight of the total toothpaste composition. Formulations containing one or more gumming ingredients at about 1.5% by weight of the total toothpaste composition allows the unit to have a nice jelly-like mouthfeel that is easy to bite, breaks up and dissolves easily, and easy to work into a foam. Formulations containing one or more gumming ingredients at about 2.25% of the total toothpaste composition result in the unit having a bit firmer feel than formulations containing carrageenan at about 1.5% of the total toothpaste composition, requiring a few more bites (i.e., one to two more bites) to work the unit into a foam.

One or more surfactant ingredients may also be included in the toothpaste composition. The one or more surfactant ingredients are intended to facilitate the dispersion of the toothpaste throughout the oral cavity in order to enhance the cleaning effect. The foaming effect produced by the surfactant ingredients are also beneficial in cleaning teeth by removing any debris and giving a feeling of cleanness. In the preferred embodiment, the one or more surfactant ingredients are selected from the group consisting of sodium lauryl sulfate, cocamidopropyl betaine, sodium lauroyl glutamate, sodium lauroyl sarcosinate, mixtures containing glycolipids sold commercially by Evonik Operations GmbH under the trademarked brand name Rheance®-1, and combinations thereof. In the most preferred formulation, the surfactant ingredients include sodium lauryl sulfate. In certain embodiments, the amount of surfactant ingredients exist in a range of about 0.5% to about 4.0% by weight of the total toothpaste composition. The more preferred range of surfactant ingredients present is about 1.0% to about 3.5% by weight of the total toothpaste composition. The most preferred amount of surfactant ingredients present in the final toothpaste composition is between about 1.5% to about 2.0% by weight of the total toothpaste composition. Sodium lauryl sulfate is the most preferred foaming agent due to the rate and volume at which it is capable of foaming, making it easier for the single-dose oral hygiene unit to be worked into a foam.

The toothpaste composition may include one or more humectant ingredients. The one or more humectant ingredients are capable of preventing loss of water, and subsequent hardening of the toothpaste composition within a container or when it is exposed to air. In the preferred embodiment, the one or more humectant ingredients are selected from the group consisting of glycerin, propanediol, propylene glycol, PEG-8, PEG-6, PEG-400, PEG-12, sorbitol, xylitol, and combinations thereof. In the preferred embodiment, the amount of humectant ingredients present in the final toothpaste composition is about 55% to about 75% by weight of the total toothpaste composition. The more preferred range of humectant ingredients present is about 63% to about 70%. More specifically, in a preferred embodiment, there may be between about 30% to about 45% of glycerin by weight in the total toothpaste composition. In a more preferred embodiment, there may be between about 29.60% to about 39.25% of glycerin by weight in the total toothpaste composition. In the most preferred embodiment, there is about 37.60% of glycerin by weight in the total toothpaste composition. In a preferred embodiment, there may be between about 8% to about 17% of PEG-8 by weight in the total toothpaste composition. In a more preferred embodiment, there may be between about 10% to about 15% of PEG-8 by weight in the total toothpaste composition. In the most preferred embodiment, there is about 10% of PEG-8 by weight in the total toothpaste composition. PEG-8 reduces the stickiness of the unit, therefore, formulations containing 15% PEG-8 are generally less sticky compared to formulations that contain, for example, 10% PEG-8. In certain embodiments, it may be advantageous to have a slightly sticky unit so that the outer surface of the unit may easily retain any coating agent that may be applied, as will be described in further detail below. In a preferred embodiment, there may be between about 0.01% to about 4% of xylitol by weight in the total toothpaste composition. In the most preferred embodiment, there is about 1% of xylitol by weight in the total toothpaste composition. In a preferred embodiment, there may be between about 8% to about 17% of propanediol by weight in the total toothpaste composition. In a more preferred embodiment, there may be between about 10% to about 15% of propanediol by weight in the total toothpaste composition. In the most preferred embodiment, there is about 15% of propanediol by weight in the total toothpaste composition.

One or more abrasive ingredients may be included in the toothpaste composition. The one or more abrasive ingredients are capable of abrading, grinding, or polishing the surface of teeth without scratching or damaging the teeth. The degree of abrasiveness is proportional to the hardness, morphology, and concentration of the particular abrasive ingredient selected. In the preferred embodiment, one or more abrasive ingredients are selected from the group consisting of hydrated silica, calcium carbonate, baking soda, and combinations thereof. In the preferred embodiment, the amount of abrasive ingredient present in the final toothpaste composition is about 8% to about 17% by weight of the total toothpaste composition. The more preferred range of abrasive ingredient present is about 10% to about 15%. In the most preferred embodiment, there is about 15% of abrasive ingredient by weight in the total toothpaste composition, and the abrasive ingredient is hydrated silica. Certain abrasive ingredients, for example hydrated silica, may also have inherent physical properties that make it a suitable gumming ingredient as well. For this reason, compositions that contain a higher percentage of abrasive ingredients may include less of a particular gumming ingredient.

The toothpaste composition also includes one or more flavoring and/or coloring ingredients. The one or more flavoring and/or coloring ingredients are capable of masking unpleasant smells or tastes of the other ingredients that are present in the final toothpaste composition. The flavoring ingredients, or sweeteners, are typically solubilized and dispersed through the toothpaste composition by the one or more surfactant ingredients responsible for foaming the toothpaste within the oral cavity of the user. Non-limiting examples of flavors that can be used in final toothpaste composition include natural flavoring agents, artificial flavoring agents, artificial extracts, natural extracts, and combinations thereof. The one or more coloring ingredients are chosen to create a desirable appearance (i.e., glittery-shimmer appearance) that consumers may associate with a particular flavoring ingredient (i.e., mint). In the preferred embodiment, the one or more flavoring and/or coloring ingredients are selected from the group consisting of sodium saccharinate (saccharin), stevia, aspartame, sucralose, peppermint oil, menthol, FD&C colors, mica, a mixture of calcium aluminum borosilicate, titanium dioxide, silicon dioxide, tin oxide sold commercially by Merck KGaA under the trademarked brand name Ronastar® Frozen Jewel, and combinations thereof. In the preferred embodiment, the amount of flavoring and/or coloring ingredients present in the final toothpaste composition are about 0.10% to about 0.76% by weight of the total toothpaste composition. In the most preferred embodiment, flavoring and/or coloring ingredients present are about 0.76%. More specifically, in a preferred embodiment, there may be about 0.1% of a sweetener ingredient, most preferably sodium saccharinate, by weight in the total toothpaste composition. In a preferred embodiment, there may be about 0.15% of FD&C color by weight in the total toothpaste composition. In a preferred embodiment, there may be between about 0.5% to about 1.5% of peppermint oil by weight in the total toothpaste composition.

The toothpaste composition may include one or more active ingredients chosen to facilitate the mitigation, prevention, and/or treatment of cavities, tooth sensitivity, tartar buildup, staining, and/or enamel damage. In some formulations the one or more active ingredients are selected from the group consisting of whitening agents, anticavity agents, tartar control agents, sensitivity relief agents, and combinations thereof. The toothpaste composition may also include solvents and preservatives. In a preferred formulation, the solvent used to dissolve the ingredients together is water, but in alternative embodiments may be any other solvent capable of dissolving and mixing the ingredients together to form a homogenous toothpaste composition necessary for the method of manufacturing the oral hygiene units described herein. In a preferred formulation, there may be between about 12% to about 17% of solvent by weight in the total toothpaste composition. In a more preferred formulation, there is about 15% of solvent by weight in the total toothpaste composition. In a preferred formulation, the preservative ingredient may be benzyl alcohol, sodium benzoate, and combinations thereof, but may be any other preservative ingredients capable of preventing the growth of micro-organisms in the toothpaste composition. In a preferred formulation, there may be between about 0.25% to about 0.75% of preservative ingredient by weight in the total toothpaste composition. In a more preferred formulation, there is about 0.5% of preservative ingredient by weight in the total toothpaste composition. When the formulation includes a whitening agent, the whitening agent may be selected from the group consisting of hydrogen peroxide, carbamide peroxide, and combinations thereof, but may be any other whitening agent capable of removing extrinsic stains on the surface of teeth, thereby lightening the teeth color. When the formulation includes an anticavity agent, the anticavity agent may be selected from a group consisting of sodium fluoride, stannous fluoride, sodium monofluorophosphate, sodium tripolyphosphate, hydroxyapatite, nano-hydroxyapatite, and combinations thereof. In embodiments comprising sodium fluoride, the sodium fluoride may be present between about 0.24% to about 0.25% by weight in the total toothpaste composition. In embodiments comprising stannous fluoride, the stannous fluoride may be present about 0.54% by weight in the total toothpaste composition. In embodiments comprising sodium monofluorophosphate, the sodium monofluorophosphate may be present about 0.76% by weight in the total toothpaste composition. When the formulation includes a tartar control agent, the tartar control agent may be zinc citrate, but may be any other tartar control agent capable of delaying calcification of plaque on the surface of teeth. When the formulation includes a sensitivity relief agent, the sensitivity relief agent may be potassium nitrate, but may be any other sensitivity relief agent capable of creating a protective layer over the pores of the enamel of teeth that lead to the nerves, preventing a sudden sensation of cold or heat from triggering discomfort.

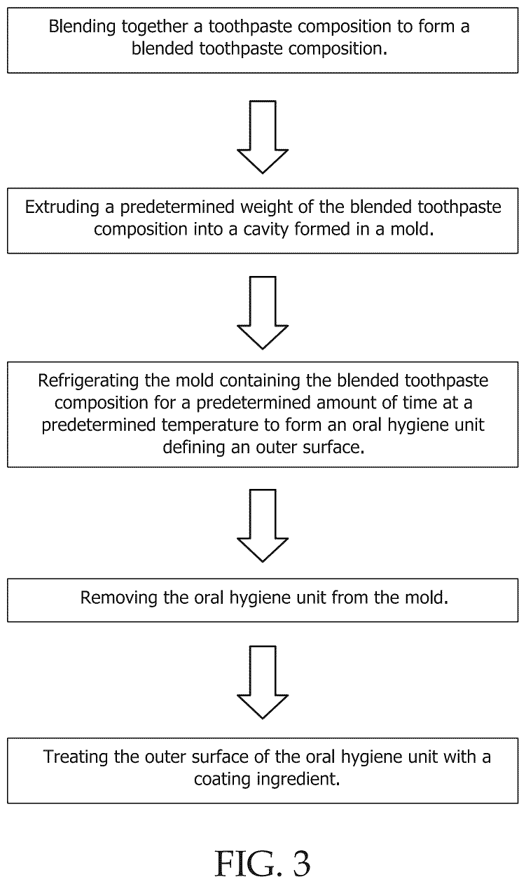

The following examples and formulations demonstrate the method of blending a toothpaste composition for forming single-dose oral hygiene units. FIG. 3 of the application illustrates a flow chart of the steps for blending, extruding, and refrigerating the blended toothpaste composition, and treating the resulting oral hygiene unit with a coating ingredient to prevent the units from sticking to one another or the container in which they are stored.

### 1. Blending the Toothpaste Composition

The aforementioned ingredients of the toothpaste composition are blended together in specific step-wise fashion allowing the toothpaste composition to be poured in a cavity of a mold tray and form a homogenous, single-dose oral hygiene unit. In certain embodiments, see Example 1, the ingredients contained in the toothpaste composition may be broken into eight (8) phases, A1, A2, B, C, D, E, F, and G. In other embodiments typically containing one or more active ingredients, see Example 2, the ingredients contained in the toothpaste composition may be broken into ten (10) phases, A1, A2, B, C, D, E, F, G, H, and I. Each phase may be defined by one or more ingredients, for example, typically A1 is defined to include one or more gumming ingredients and one or more humectant ingredients. Examples are provided below to identify which ingredients are defined by each blending phase.

### Example 1—Formulation 31B

| Ingredient | % by weight | Blending Phase |
|---|---|---|
| Glycerin | 39.25 | A1 |
| Carrageenan | 1.50 | |
| PEG-8 | 10.00 | A2 |
| Water | 15.00 | B |
| Xylitol | 1.00 | |
| Sodium Saccharinate | 0.10 | |
| FD&C color | 0.15 | |
| Propanediol | 15.00 | C |
| Hydrated Silica | 15.00 | D |
| Benzyl Alcohol | 0.50 | E |
| Peppermint Oil | 0.50 | F |
| Sodium Lauryl Sulfate | 2.00 | G |
| Total | 100.00 | |

### Example 2—Formulation 24E

| Ingredient | % by weight | Blending Phase |
|---|---|---|
| Glycerin | 37.10 | A1 |
| Carrageenan | 2.25 | |
| PEG-8 | 10.00 | A2 |
| Water | 15.00 | B |
| Xylitol | 1.00 | |
| Erylite Stevia | 0.10 | |
| Sodium Fluoride | 0.30 | |
| CI 42090 1% | 0.15 | |
| Propanediol | 15.00 | C |
| Hydrated Silica | 15.00 | D |
| Benzyl Alcohol | 0.50 | E |
| Peppermint Oil | 0.60 | F |
| Sodium Lauryl Sulfate | 0.50 | G |
| Rheance ®-1 | 2.00 | H |
| Ronacare ® Frozen Jewel | 0.50 | I |
| Total | 100 | |

In the preferred method of manufacture, the toothpaste composition is produced using a specific set of blending instructions. In the Example 1—Formulation 31B and Example 2—Formulation 24E above, the first step is to thoroughly disperse the ingredients of the A1 phase. Next, the ingredients of the A2 phase are added, under agitation, to the thoroughly dispersed ingredients from the A1 phase. Agitation is the process of mixing until the ingredients are fully dissolved. Each of the ingredients of the B phase are dissolved separately in hot water until the resulting mixture is homogenous. Once the B phase ingredients are dissolved in the water, they are added into the mixture of ingredients from the A1 and A2 phases. Then the A1-A2-B mixture is heated at least to about eighty-five degrees) (85° Celsius, while maintaining rapid agitation. The temperature shall remain, at a minimum, about 85 degrees Celsius for the remainder of the blending instructions. The ingredients of the C phase are added to the mixture under stirring. Next, the ingredients of the D phase are added to the mixture in small fractions, preferably under rapid shearing. As used herein, rapid shearing shall be understood to be a mechanical process that uses fine blades to slice through a mixture to break up any lumps. Shearing may be accomplished by various machines in the industry containing rotors and blades, for example, a homogenizer. Stirring shall be maintained after shearing until a lump-free mixture is obtained. The remaining ingredients (the ingredients from the E, F, and G phases, or from the E, F, G, H, and I phases) are added to the mixture, one at a time, under rapid agitation. Once the final ingredients have been added, the temperature is maintained at a minimum of at least 85 degrees Celsius, and the mixture is stirred until a smooth, lump-free composition is obtained.

2. Extruding the Final, Blended Toothpaste Composition into Molds

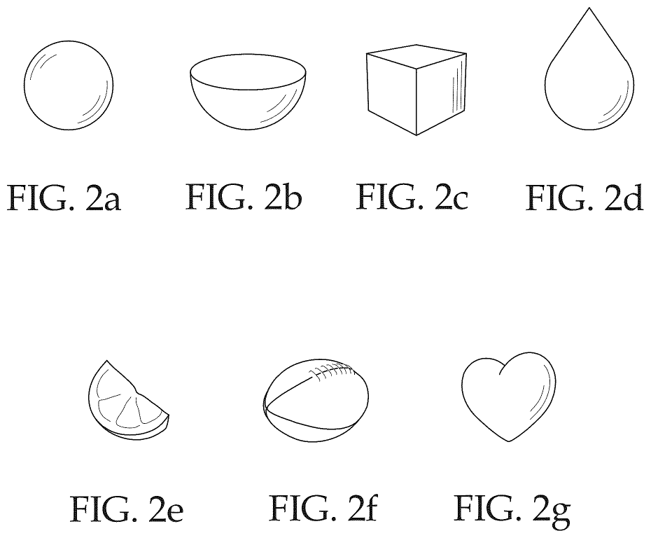

Once the blending instructions above are complete, the smooth, lump-free composition is precisely deposited into molds via nozzles. The nozzles are calibrated to control the exact amount of formula, ensuring uniform sizes and shapes. The mold tray may include any number of cavities and the cavities may define any shape such as demonstrated in FIGS. 2A-2G. In a preferred method, the cavities define a hemi-spherical shape, but may define other shapes, such as full spheres, cubes, strips, hearts, tear drops, wedges, cartoon characters, shapes that represent sporting items such as footballs, soccer balls, tennis balls, or golf balls. In one embodiment, the extruder is configured with two rows of ten extruder nozzles such that twenty cavities may be filled at one time, and then the conveyor is moved along, and the extruder nozzles fill another twenty cavities with the blended toothpaste composition. While the shape and number of cavities in any particular mold may change, the amount, in mass and/or volume, of final blended toothpaste composition extruded into each cavity is about 0.75 grams to 2.50 grams of final, blended toothpaste composition per cavity. These amounts are defined by the American Dental Association as the appropriate amount of toothpaste per brushing event.

### 3. Refrigerating the Molds Containing the Final, Blended Toothpaste Composition

Once all the cavities are filled with the final, blended toothpaste composition, the molds are refrigerated until the final, blended toothpaste composition within each cavity solidifies into a homogenous jelly-like unit. In the preferred method, the molds are refrigerated at a temperature of about minus one degree (−1°) Celsius for between about four and about twelve minutes. Once the final, blended toothpaste composition forms a homogenous unit each unit is removed from the mold tray and optionally go through the refrigeration cycle once again, this time outside of the mold tray for up to another twelve minutes.

### 4. Coating Treatment of the Oral Hygiene Units

The final step in the manufacturing of the oral hygiene units is to treat the outer surface of each unit so that two or more units resist sticking together when packaged and placed in the final container. As used herein, the term “treating” includes spraying, dusting, sprinkling, coating, tumbling, pouring in, or by any other suitable means, such as by use of a depositor, sifter, or powder bed. The coating treatment also allows the user to easily manipulate the units without sticking to the fingers of the user. In the preferred method, the surface treatment is performed by tumbling the units in a rotary drum with the coating ingredient. The coating ingredient is typically selected from the group consisting of citrus fiber powder, carnauba wax, and combinations thereof. In the preferred method, the oral hygiene units are tumbled and lightly coated with a citrus fiber powder until the outer surface of each unit is lightly and evenly coated. Citrus fiber powder improves the texture and reduces stickiness of the units. In alternative methods, the oral hygiene units are glazed and lightly coated with a carnauba wax. Carnauba wax provides a polished finish on the outer surface and increases the melting temperature of the units. Once the oral hygiene units have cooled and dried, they are packaged in a container and are ready for distribution.

FIG. 1 shows a conventional toothbrush 40 alongside a plurality of oral hygiene units 20 which are capable of being stored in a container 30 because the outer surface of each unit 20 is treated to resist sticking together or sticking to the container. The container 30 may take any shape or size and is preferably formed for easy access and convenience in storage.

The illustrations and examples provided herein are for explanatory purposes and are not intended to limit the scope of the appended claims.

We claim:

1. A single-dose oral hygiene unit of a toothpaste composition, the oral hygiene unit comprising:
a toothpaste composition in the nature of a unit defining an outer surface, the toothpaste composition including: one or more humectant ingredients, one or more gumming ingredients, and one or more surfactant ingredient; and
one or more coating ingredients on the outer surface of the unit to prevent two or more units from sticking together.

2. The oral hygiene unit of claim 1, wherein the one or more humectant ingredients are present at about 63% to about 70% by weight of the toothpaste composition.

3. The oral hygiene unit of claim 1, wherein the one or more gumming ingredients are present at about 1.0% to about 3.5% by weight of the toothpaste composition.

4. The oral hygiene unit of claim 1, wherein the one or more surfactant ingredients are present at about 1.0% to about 3.5% by weight of the toothpaste composition.

5. The oral hygiene unit of claim 1, wherein the one or more humectant ingredients are selected from the group consisting of glycerin, propanediol, propylene glycol, PEG-8, PEG-6, PEG-400, PEG-12, sorbitol, xylitol, and combinations thereof.

6. The oral hygiene unit of claim 1, wherein the one or more gumming ingredients are selected from the group consisting of carrageenan, xanthan gum, guar gum, arabic gum, carob bean gum, cellulose gum, and combinations thereof.

7. The oral hygiene unit of claim 1, wherein the surfactant ingredient is selected from the group consisting of sodium lauryl sulfate, cocamidopropyl betaine, sodium lauroyl glutamate, sodium lauroyl sarcosinate, Rheance®—1, and combinations thereof.

8. The oral hygiene unit of claim 1, wherein the coating ingredient is selected from the group consisting of citrus powder, carnauba wax, and combinations thereof.

9. The oral hygiene unit of claim 1, wherein the toothpaste composition further includes an active ingredient selected from the group consisting of one or more whitening agents, one or more anticavity agents, one or more tartar control agents, one or more sensitivity relief agents, and combinations thereof.

10. The oral hygiene unit of claim 9, wherein the one or more whitening agents are selected from the group consisting of hydrogen peroxide, carbamide peroxide, and combinations thereof.

11. The oral hygiene unit of claim 9, wherein the one or more anticavity agents are selected from the group consisting of sodium fluoride, stannous fluoride, sodium monofluorophosphate, sodium tripolyphosphate, hydroxyapatite, and combinations thereof.

12. The oral hygiene unit of claim 9, wherein the one or more tartar control agents is zinc citrate.

13. The oral hygiene unit of claim 9, wherein the one or more sensitivity relief agents is potassium nitrate.

14. A method of forming an oral hygiene unit of a toothpaste composition, the method comprising:
blending together a toothpaste composition to form a blended toothpaste composition;
extruding a predetermined weight of the blended toothpaste composition into a cavity formed in a mold;
refrigerating the mold containing the blended toothpaste composition for a predetermined period of time at a predetermined temperature to form an oral hygiene unit defining an outer surface;
removing the oral hygiene unit from the mold; and
treating the outer surface of the oral hygiene unit with a coating ingredient.

15. The method of claim 14, wherein the predetermined weight of blended toothpaste composition is between about 0.75 grams to about 2.25 grams.

16. The method of claim 14, wherein the cavity forms a shape, the shape selected from a group consisting of hemi-spheres, spheres, cubes, strips, hearts, cartoon characters, and shapes that represent sporting items such as footballs, soccer balls, tennis balls, and golf balls.

17. The method of claim 14, further comprising the step of packaging a plurality of oral hygiene units into a container.

18. The method of claim 14, wherein the predetermined period of time for refrigerating the mold containing the blended toothpaste composition is between about four minutes and twelve minutes.

19. The method of claim 14, wherein the predetermined temperature for refrigerating the mold containing the blended toothpaste composition is between minus six Celsius and positive four degrees Celsius.

20. The method of claim 14, wherein the coating ingredient is selected from a group consisting of citrus powder, carnauba wax, and combinations thereof.
